(19) United States
(12) Patent Application Publication
(10) Pub. No.: US 2023/0011085 A1
(43) Pub. Date: Jan. 12, 2023

(54) METHOD AND SYSTEM FOR DETERMINING A CNV PROFILE FOR A TUMOR USING SPARSE WHOLE GENOME SEQUENCING

(71) Applicant: KONINKLIJKE PHILIPS N.V., EINDHOVEN (NL)

(72) Inventors: Jie WU, Cambridge, MA (US); Yee Him CHEUNG, Boston, MA (US); Nevenka Dimitrova, Pelham Manor, NY (US)

(21) Appl. No.: 17/779,624

(22) PCT Filed: Dec. 3, 2020
(86) PCT No.: PCT/EP2020/084403

§ 371 Date: May 25, 2022

Related U.S. Application Data
(60) Provisional application No. 62/947,023, filed on Dec. 12, 2019.

(51) Int. Cl. G16B 20/10

(52) U.S. Cl. CPC G16B 20/10

(57) ABSTRACT

A method (100) for determining a copy number variation (CNV) profile, comprising: (i) receiving (110) sparse genome sequencing data; (ii) determining (120) an unadjusted CNV profile; (iii) normalizing (130) the unadjusted CNV profile; (iv) receiving (140) a range for possible ploidy and for a possible contamination rate; (v) determining (150) adjusted segmentation values for the CNV profile; (vi) determining (160) a plurality of adjustment scores comprising a distance between an adjusted segmentation value and a closest whole integer for a CNV call; (vii) comparing (170) the determined plurality of adjustment scores to one or more predetermined factors for selecting a CNV profile best fit; (viii) selecting (180) one of the plurality of adjustment scores as a best fit for the copy number variation profile of the tumor cells of the tumor; (ix) generating (190) an adjusted CNV profile report; and (x) reporting (192) the generated adjusted CNV profile report.

### FIELD OF THE DISCLOSURE

The present disclosure is directed generally to methods and systems for characterizing an accurate copy number variation (CNV) profile of tumor cells from a tumor sample using sparse whole genome sequencing.

### BACKGROUND

Copy number variation (CNV) is a class of somatic mutational events that is of importance clinically from a diagnostic, prognostic, and therapeutic point of view. As just one example, CNV data can be an essential component of cancer diagnosis and prognosis. CNV data can also be used to guide targeted therapy and risk-directed therapy. Indeed, the clinical utility of copy number information is widely acknowledged in cancers such as acute myeloid leukemia and breast cancer, and is increasingly being recognized for its importance in other cancer disease entities. For example, CNV analysis can also be used to uncover clinically-actionable genetic aberrations in other cancers such as in melanoma, non-small-cell lung carcinoma and colorectal cancer.

Unfortunately, determination of CNV information for tumor cells can be complicated. Clinical tumor samples are typically mixtures of tumor cells and other cells such as stromal cells, and thus a deconvolution is necessary for a better understanding of the tumor. To produce more accurate CNV calls, tumor cell contamination must be accounted for by adjusting the initial CNV results to absolute copy numbers. Accounting for purity can make CNV detection more accurate.

### SUMMARY OF THE DISCLOSURE

There is a continued need for methods and systems that characterize an accurate copy number variation profile of tumor cells from a tumor sample using faster and more cost-effective methods. The present disclosure is directed to inventive methods and systems for characterizing copy number variation for a tumor cell using sparse whole genome sequencing. Various embodiments and implementations herein are directed to a system and method that determines, from sparse genome data, an initial unadjusted CNV profile comprising a plurality of CNV calls for a plurality of chromosomes. The system then normalizes that unadjusted CNV profile to a mean value of 1. According to an embodiment, the system comprises a predetermined range for ploidy for the genome data, and a predetermined range for a contamination rate for the genome data. The system uses that information to determine adjusted segmentation values for the plurality of CNV calls, and then determines a plurality of adjustment scores each comprising a distance between the adjusted segmentation values and closest whole integers for a CNV. The determined plurality of adjustment scores are compared to one or more factors that influence the selection of a CNV profile best fit, such as CNV profiles previously observed and preferred by a clinician, and/or ploidy and contamination distributions from previous data, among other possible factors. Based on that comparison, the system selects one of the plurality of adjustment scores as a best fit for the copy number variation profile of the tumor cells of the tumor. According to an embodiment, the system generates an adjusted CNV profile report using the selected best fit adjustment score and provides the generated adjusted CNV profile report, such as to a user, user interface, or other display or system.

Generally, in one aspect, is a method for determining a copy number variation (CNV) profile of target cells from a sample using a CNV profiling system. The method includes: (i) receiving sparse genome sequencing data comprising sequencing from both target and non-target cells from the sample; (ii) determining, from the received sparse genome data, an unadjusted CNV profile comprising a plurality of CNV calls for a plurality of chromosomes; (iii) normalizing the unadjusted CNV profile; (iv) receiving a range for possible ploidy for the CNV profile, and/or receiving a range for a possible contamination rate for the CNV profile; (v) determining, using the received ploidy range and/or received contamination rate range, adjusted segmentation values for the plurality of CNV calls; (vi) determining a plurality of adjustment scores comprising a distance between adjusted segmentation values and closest whole integers for a CNV profile; (vii) comparing the determined plurality of adjustment scores to one or more predetermined factors for selecting a CNV profile best fit; (viii) selecting, based at least in part on the comparison, one of the plurality of adjustment scores as a best fit for the copy number variation profile of the tumor cells of the tumor; (ix) generating, using the selected a best fit adjustment score, an adjusted CNV profile report; and (x) reporting the generated adjusted CNV profile report.

According to an embodiment, the method further includes identifying, using the CNV profile report, one or more causal CNVs and providing an intervention based on the identified one or more causal CNVs.

According to an embodiment, the unadjusted CNV profile is normalized to a mean value of one.

According to an embodiment, the range for possible ploidy for the CNV profile and the range for a possible contamination rate for the CNV profile is received from a user of the CNV profiling system.

According to an embodiment, determining adjusted segmentation values for the plurality of CNV calls comprises the equation S_{adj} = P(S - C)/(1 – C) where S_{adj} is an adjusted segmentation value for a CNV segment, P is a ploidy value from the range for possible ploidy, C is a contamination rate value from the range for possible contamination rate, and S is a segmentation value before adjustment.

According to an embodiment, determining a plurality of adjustment scores comprises the equation

$$\text{D} = {{\sum }_{i = 1}^{n} \left({S}_{a d j}^{i} - r o u n d \left({S}_{a d j}^{i}\right)\right)}^{2}$$

where D is a calculated distance between an adjusted segmentation value (S_{adj}) and a closest whole integer, S_{adj} is an adjusted segmentation value of an ith segment, and n is a number of autosome segments.

According to an embodiment, one of the one or more predetermined factors for selecting a CNV profile best fit is a CNV profile previously observed by a user, a ploidy value or range previously observed by a user, a contamination value or range previously observed by a user, and/or ploidy or contamination information from a previous analysis.

According to an embodiment, the target cells are tumor cells.

According to a second aspect is a system for determining a copy number variation (CNV) profile of target cells from a sample. The system includes: sparse genome sequencing data comprising sequencing from both target and non-target cells from the sample; a processor configured to: (i) determine, from the received sparse genome data, an unadjusted CNV profile comprising a plurality of CNV calls for a plurality of chromosomes; (ii) determine, using a received ploidy range and/or received contamination rate range, adjusted segmentation values for the plurality of CNV calls; (iii) determine a plurality of adjustment scores comprising a distance between adjusted segmentation values and closest whole integer for a CNV profile; (iv) compare the determined plurality of adjustment scores to one or more predetermined factors for selecting a CNV profile best fit; (v) select, based at least in part on the comparison, one of the plurality of adjustment scores as a best fit for the copy number variation profile of the tumor cells of the tumor; and (vi) generate, using the selected a best fit adjustment score, an adjusted CNV profile report; and a user interface (840) configured to provide the generated report.

According to an embodiment, the user interface is further configured to receive a range for possible ploidy for the CNV profile, and/or receive a range for a possible contamination rate for the CNV profile.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for determining a copy number variation profile, in accordance with an embodiment.

FIG. 2A is an example of an initial unadjusted CNV profile, in accordance with an embodiment.

FIG. 2B is an example of an initial unadjusted CNV profile, in accordance with an embodiment.

FIG. 2C is an example of an initial unadjusted CNV profile, in accordance with an embodiment.

FIG. 3A is an example of an adjusted CNV profile, in accordance with an embodiment.

FIG. 3B is an example of an adjusted CNV profile, in accordance with an embodiment.

FIG. 3C is an example of an adjusted CNV profile, in accordance with an embodiment.

FIG. 4A is an example of a best fit adjusted CNV profile, in accordance with an embodiment.

FIG. 4B is an example of a best fit adjusted CNV profile, in accordance with an embodiment.

FIG. 4C is an example of a best fit adjusted CNV profile, in accordance with an embodiment.

FIG. 5A is a preferred fit graph, in accordance with an embodiment.

FIG. 5B is an adjustment score graph, in accordance with an embodiment.

FIG. 6A is a preferred fit graph, in accordance with an embodiment.

FIG. 6B is an adjustment score graph, in accordance with an embodiment.

FIG. 7A is a preferred fit graph, in accordance with an embodiment.

FIG. 7B is an adjustment score graph, in accordance with an embodiment.

FIG. 8 is a comparison of an unadjusted CNV profile (top panel) and a generated best fit CNV profile (bottom panel), in accordance with an embodiment.

FIG. 9 is an example of an adjustment score graph, in accordance with an embodiment.

FIG. 10 is an example of a preferred fit graph, in accordance with an embodiment.

FIG. 11A is an example of an adjustment score graph, in accordance with an embodiment.

FIG. 11B is an example of a preferred fit graph, in accordance with an embodiment.

FIG. 11C is a generated best fit CNV profile, in accordance with an embodiment.

FIG. 12 is a schematic representation of a system for determining a copy number variation profile, in accordance with an embodiment.

### DETAILED DESCRIPTION OF EMBODIMENTS

The present disclosure describes various embodiments of a system and method to determining a copy number variation profile of tumor cells from a tumor sample using sparse genome data. More generally, Applicant has recognized and appreciated that it would be beneficial to provide a method and system that can characterize an accurate copy number variation profile of tumor cells using faster and more cost-effective methods. The system uses sparse genome data to generate an unadjusted CNV profile comprising a plurality of CNV calls for a plurality of chromosomes, which can then be normalized. According to an embodiment, the system comprises a range for ploidy for the genome data, and a range for a contamination rate for the genome data. The system uses that information to determine an adjusted segmentation value for at least one of the plurality of CNV calls, and then determines a plurality of adjustment scores comprising distances between the adjusted segmentation value and different closest whole integers for a CNV profile. The determined plurality of adjustment scores are compared to one or more factors that influence the selection of a CNV profile best fit, such as CNV profiles preferred by a clinician, and/or ploidy and contamination distributions from previous data, among other possible factors. Based on that comparison, the system selects one of the plurality of adjustment scores as a best fit for the copy number variation profile of the tumor cells of the tumor. According to an embodiment, the system generates an adjusted CNV profile report using the selected a best fit adjustment score and provides the generated adjusted CNV profile report, such as to a user, user interface, or other display or system.

According to an embodiment, sparse whole genome sequencing has been overlooked by research and healthcare communities. Although sparse whole genome sequencing is a cost-effective technique to retrieve genome-wide cytogenetic information, there is no CNV-based pipeline for clinical use with sparse whole genome sequencing. Indeed, CNV information, unlike smaller variants such as single nucleotide variants, can be retrieved via sparse whole genome sequencing data. The nature of this approach makes it highly cost effective (including an order of magnitude cheaper or more), and it also yields much more uniform read distribution than whole exome sequencing and covers the whole genome to enable a larger spectrum. It is also fare more sensitive than array-based methods.

One of the many advantages of the methods and systems described or otherwise envisioned herein is that they enable non-tumor cell contamination analysis and adjustment without a control. According to an embodiment, the system only utilizes the measured copy number data for purity estimation, and no other variant data (such as single nucleotide variant) is utilized.

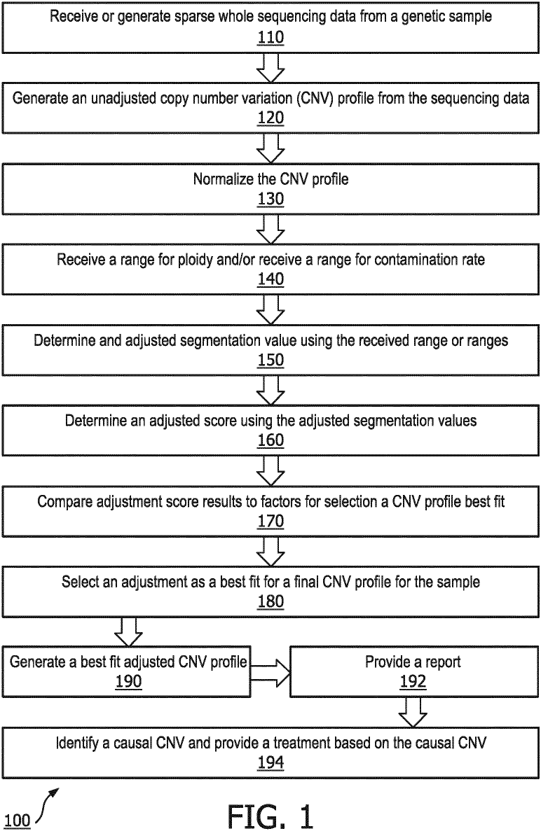

Referring to FIG. 1, in one embodiment, is a flowchart of a method 100 for determining copy number variation of tumor cells using sparse genome data and a CNV profiling system. The CNV profiling system may be any of the systems described or otherwise envisioned herein, and may comprise any of the components described or otherwise envisioned herein.

At step 110 of the method, the CNV profiling system receives or generates sparse whole genome sequencing data from a sample. According to an embodiment, sparse whole genome sequencing data comprises much less information than high-depth next-generation whole genome sequencing data. For example, the sparse whole genome sequencing data may comprise fewer than 10 million reads for a human genome, comprising approximately 0.1x coverage of that genome.

According to an embodiment, the sample is a tumor sample from an individual such as a patient or other person, and comprises both tumor and non-tumor cells. According to an embodiment, the sample can be any genetic sample from any organism, including humans, pathogenic and non-pathogenic organisms, and many others. It is recognized that there is no limitation to the source of the genetic sample.

According to an embodiment, the CNV profiling system comprises a DNA sequencing platform configured to obtain sparse whole genome sequencing data from the genetic sample. The sequencing platform can be any sequencing platform, including but not limited to any system described or otherwise envisioned herein. A sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner. According to an embodiment, the CNV profiling system receives the sparse whole genome sequencing data from the genetic sample. For example, the CNV profiling system may be in communication or otherwise receive the sequencing data from a database comprising one or more genetic samples. According to one embodiment, the generated and/or received sparse sequencing data may comprise a complete or mostly complete genome, or may be a partial genome.

The generated and/or received sparse whole genome sequencing data may be stored in a local or remote database for use by the CNV profiling system. For example, the CNV profiling system may comprise a database to store the sequencing data for the genetic sample, and/or may be in communication with a database storing the sequencing data. These databases may be located with or within the CNV profiling system or may be located remote from the CNV profiling system, such as in cloud storage and/or other remote storage.

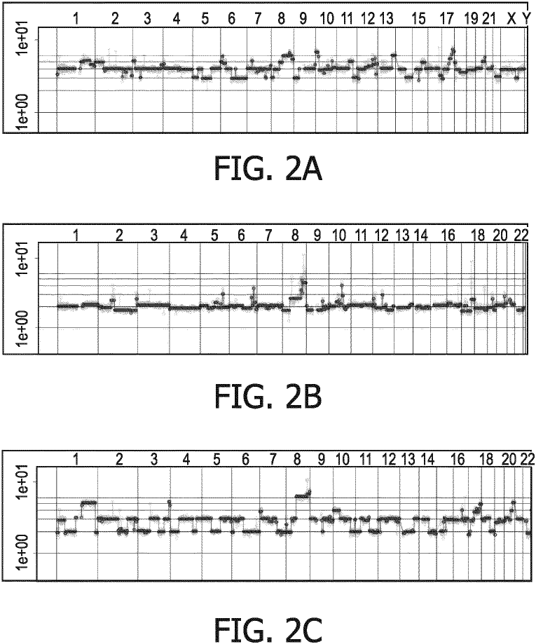

At step 120 of the method, the CNV profiling system determines an initial unadjusted CNV profile from the sparse genome data, comprising a plurality of CNV calls for a plurality of chromosomes or other genomic regions or breakdowns. The initial unadjusted CNV profile determination may be performed using any of a wide variety of different CNV analysis platforms or methods. Referring to FIGS. 2A, 2B, and 2C are examples of initial unadjusted CNV profiles determined or received by the CNV profiling system.

According to one embodiment, the copy number profile for a DNA sample comprising a number of cells from a tumor section will be composed of a component from the normal diploid cells and the tumor cells within that sample. Some or many of the normal diploid cells in the sample may be, for example, stromal cells among other types of cells. As the tumor cells are likely to be mostly from a single copy number clone, the normal and tumor components of the copy number profile can be separated and an estimate of the percentage of normal cells in the tumor section sequenced. The total copy number profile will be composed of a variable copy number tumor profile plus a constant normal cell profile. As described below, after subtracting the constant profile it is possible to compute the best possible ploidy estimate for the remaining tumor component and then an error for that profile.

At step 130 of the method, the system normalizes the unadjusted CNV profile. The system can be configured to normalize the unadjusted CNV profile in any of a wide variety of ways and methods, including existing normalization methods. According to an embodiment, the system may be configured to normalize the unadjusted CNV profile to a mean value of one. According to an embodiment, the system may be configured by a user to normalize the unadjusted CNV profile depending on the needs or goals of the user. As described herein, these graphs of unadjusted CNV profiles comprise contamination, such as from non-cancer cells, which results in both an incorrect ploidy number as well as non-integer ploidy numbers. The analysis and deconvolution described or otherwise envisioned herein results in the correct ploidy number, including ploidy shifts upward or downward to increase accuracy.

At step 140 of the method, the system receives a range for ploidy for the genome data, and/or receives a range for a contamination rate for the genome data. These ranges can be predetermined, including being pre-programmed or otherwise received by or provided to the system. According to an embodiment, a user such as a researcher, clinician, technician, or other user can provide the ranges as a setting, selection, or other information via a user interface or any other communication method. According to an embodiment, the one or more received ranges allow the system to process the normalized unadjusted CNV profiles as described below.

The received range for ploidy (P) comprises one or more values which can be used to process the normalized unadjusted CNV profile. According to one embodiment, the received range for ploidy (P) comprises a range between and possibly including 1.5 to 4.5, although other ranges are possible. Indeed, measured ploidy can be much higher and thus can require a larger range, possibly depending on the sample and/or cause of the CNV, among other variables.

When the ploidy range or value is utilized as described herein, the value or values may be utilized with an interval that samples the range. For example, the interval for the ploidy range may be 0.1 such that sampling a range of 1.5 to 4.5 may be 1.5, 1.6, 1.7, and so on.

The received range for range for contamination rate (C) comprises one or more values which can be used to process the normalized unadjusted CNV profile. According to one embodiment, the received range for contamination rate (C) comprises a range between and possibly including 0% to 100%, with much smaller ranges possible.

When the contamination rate or value is utilized as described herein, the value or values may be utilized with an interval that samples the range. For example, the interval for the contamination rate range may be 1% such that sampling the range of 0% to 100% may comprise values of 0%, 1%, 2%, and so on.

As just one non-limiting example, a user such as a researcher or clinician may be utilizing the methods and systems described or otherwise envisioned herein to determine a copy number variation (CNV) profile of tumor cells from a tumor sample. Before or during the analysis, the user will provide an input comprising a selected or default ploidy (P) range or value, and/or an input comprising a selected or default contamination rate (C) range or value, optionally as one or more settings for the CNV profiling system. The CNV profiling system utilizes the received ranges or values to inform one or more downstream steps of the analysis.

At step 150 of the method, the system uses the received one or more ranges to determine adjusted segmentation values for the plurality of CNV calls. The adjusted segmentation values may be determined in a variety of different ways and methods. According to one embodiment, an adjusted segmentation value is determined using the unadjusted segmentation value for a CNV segment, a ploidy value based on input from step 140, and/or a contamination rate value based on input from step 140.

According to an embodiment, an adjusted segmentation value may be determined using the following equation:

$${S}_{a d j} = P ( S - C ) / ( 1 - C )$$

where S_{adj} is the adjusted segmentation value for a CNV segment, P is a ploidy value, C is a cell contamination rate value, and S is the segmentation value for the CNV segment before adjustment. However, other methods for determining an adjusted segmentation value are possible. Note that the mean value of S_adj is P, rather than normalized to 1.

According to an embodiment, an adjusted segmentation value is determined for each CNV segment using the full received ploidy range, the full received contamination rate range, or both received ranges. For example, the system may comprise or receive, such as from a user, a ploidy range of 2 to 4.5, and a contamination rate of 10% to 25%. The numbers provided in this example are provided only as possible ranges, and are not limiting. The system then determines an adjusted segmentation value for each CNV segment using a sampling rate for the ploidy range and/or for the contamination rate. For example, if the sampling rate for the ploidy is 0.1 and the received range is 2 to 4.5, the system will determine an adjusted segmentation value for each CNV segment using 2.0, 2.1, 2.2, and so on at 0.1 intervals through and including 4.5. If the sampling rate for the contamination rate is 1% and the range is 10% to 25%, the system will determine an adjusted segmentation value for each CNV segment using 10%, 11%, 12%, and so on at 1% intervals through and including 25%. There may thus be 100 s or 1000 s of determined adjusted segmentation values for a CNV segment.

The determined adjusted segmentation values for each CNV segment may be used by the system immediately or in the short-term, or may be stored in a local or remote database for future or other downstream use by the CNV profiling system.

At step 160 of the method, the system uses the adjusted segmentation values (S_{adj}) for the CNV segments to determine a plurality of adjustment scores, for example comprising a distance between the adjusted segmentation value (S_{adj}) and different closest whole integers for a CNV profile. The adjustment scores may be determined in a variety of different ways and methods. According to one embodiment, an adjustment score measures, and may allow for the minimization of, the difference between adjusted segmentation values and whole integers closest to the values. For example, according to an embodiment the system may be designed such that CNV segments are likely to be clonal, meaning they are likely to be an integer such as 1, 2, 3, etc. rather than a value such as 1.4, 2.6, 3.1, etc. This may represent an underlying assumption that CNV segments are likely to be an integer. According to an embodiment, an adjustment score may be determined for an entire CNV profile, or the adjustment score may be determined for a sub-set of the CNV profile. This may be selected or otherwise determined by a user, may be selected or determined by the system, and/or may be selected or otherwise determined by other input or selection mechanism.

According to an embodiment, an adjustment score may be determined using the following equation:

$$D = {\sum }_{i = 1}^{n} ( {S}_{a d j}^{i} - r o u n d {( {S}_{a d j}^{i} )}_{)} {}^{2}$$

where D is the calculated distance between the adjusted segmentation values (S_{adj}) and closest whole integers

$$, {S}_{a d j}^{i}$$

is the adjusted segmentation value of the ith segment, and n is the number of autosome segments in the data.

This is just one possible method or possible score function that can be used to measure the distance between the adjusted profile and the closest integer profile. According to an embodiment, the adjustment score may be determined by multiplying the above distance by the lengths of the segments to account for segment sizes, among other methods.

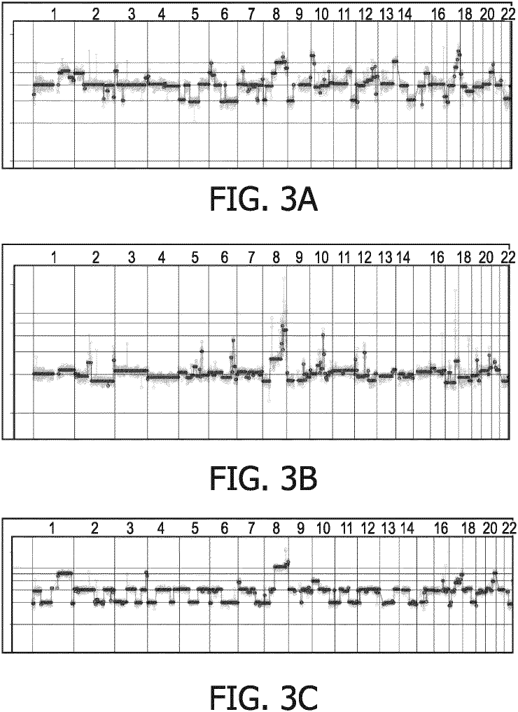

Referring to FIGS. 3A, 3B, and 3C are adjusted CNV profiles with adjusted segmentation values, prior to the best fit analysis in step 170 of the method. These profiles are rejected by the CNV profiling system as they do not comprise the best fit for CNV profile.

At step 170 of the method, the system compares the results of the adjustment score analysis to one or more factors to facilitate selection of a best fit CNV profile. This may result in one or more parameters or factors that may be used to select or influence selection of a best fit CNV profile, from among the profiles represented by the adjustment scores.

According to an embodiment, among many other factors are things such as CNV profiles or profile variables previously clinically observed by a user such as a clinician or researcher and determined to be more meaningful according to the user’s experience, including factors such as likely CNV segment integers, among others. Other factors include ploidy distributions determined from previous data or analyses, and/or contamination distributions from previous data or analyses, including but not limited to analyses where one or more parameters of the sample or analysis were similar to the current sample or analysis. In other words, the system may utilize prior information to prioritize certain solutions. For example, the system may use the distribution of contamination rate or ploidy from similar samples obtained by other techniques. In some cases, a ploidy closer to two may be more favorable as the best solution. Copy number distributions can also be used. For instance, when the predicted ploidy/contamination results in a CNV profile with all copy number bigger than two, without any lower copy numbers, the system may reject that solution (in the next step) and use another solution. Many other preferences from the clinicians can also be incorporated into the selection procedure.

At step 180 of the method, a final CNV profile is selected as a best fit for the sample, based at least in part on the one or more factors from step 170 of the method. According to an embodiment, the combination of contamination rate (C) and ploidy estimate (P) that best minimizes error in the unadjusted CNV profile, and thus generates the most likely adjusted CNV profile, is selected as the best solution. According to an embodiment, if the tumor is a single copy number clone, the segments will fall very close to integer values when the contamination rate and tumor ploidy values are correct. Thus, the combination of contamination rate and tumor ploidy values that generate an adjustment score and adjusted CNV profile with the highest likelihood of accuracy is selected.

At step 190 of the method, the system generates the best fit adjusted CNV profile using the selected adjustment. This can be performed by the system via a variety of methods and systems, to generate a final adjusted CNV profile that can be saved, reported, or otherwise stored or used by the CNV profiling system.

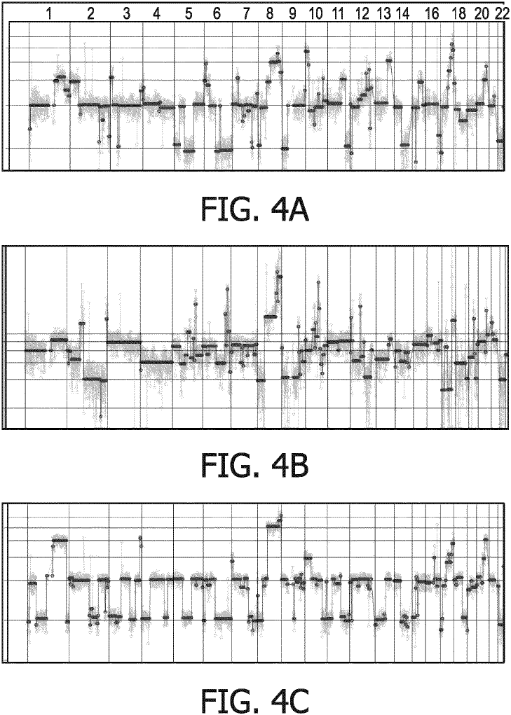

Referring to FIGS. 4A, 4B, and 4C are best fit adjusted CNV profiles generated by the CNV profiling system. These best fit adjusted CNV profiles correspond to the examples of initial unadjusted CNV profiles in FIGS. 2A/3A, 2B/3B, and 2C/3C, respectively.

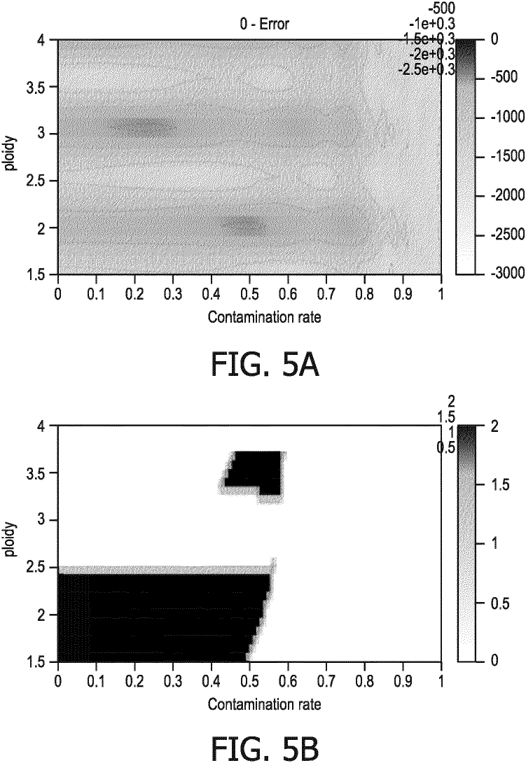

The example in FIG. 4A utilized the score graph in FIG. 5A and the preferred fit graph in FIG. 5B. Referring to FIG. 5A is a graph of adjustment score results for a given ploidy range (1.5 to 4) and a contamination range (0% to 100%). FIG. 5B is a graph of acceptable or preferred results for the given ploidy versus contamination ranges. For the example in FIG. 4A, the ploidy was shifted down by the analysis as there were no events at copy number 1 and 2.

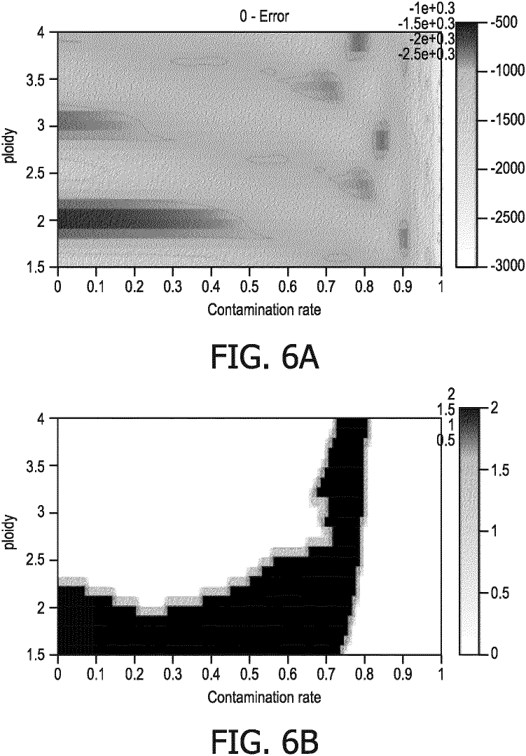

The example in FIG. 4B utilized the score graph in FIG. 6A and the preferred fit graph in FIG. 6B. Referring to FIG. 6A is a graph of adjustment score results for a given ploidy range (1.5 to 4) and a contamination range (0% to 100%). FIG. 6B is a graph of acceptable or preferred results for the given ploidy versus contamination ranges. For the example in FIG. 4B, the sample is highly contaminated at 77% with ploidy = 4. The best fit provided the improvement of at least two integer copy numbers as shown in FIG. 4B.

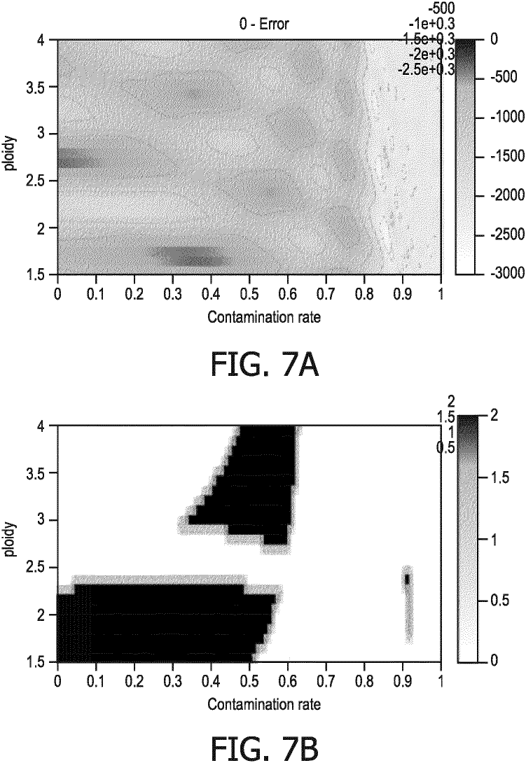

The example in FIG. 4C utilized the score graph in FIG. 7A and the preferred fit graph in FIG. 7B. Referring to FIG. 7A is a graph of adjustment score results for a given ploidy range (1.5 to 4) and a contamination range (0% to 100%). FIG. 7B is a graph of acceptable or preferred results for the given ploidy versus contamination ranges. For the example in FIG. 4C, the ploidy was shifted down by the analysis as it was unlikely that the majority of the genome would be at copy number 3.

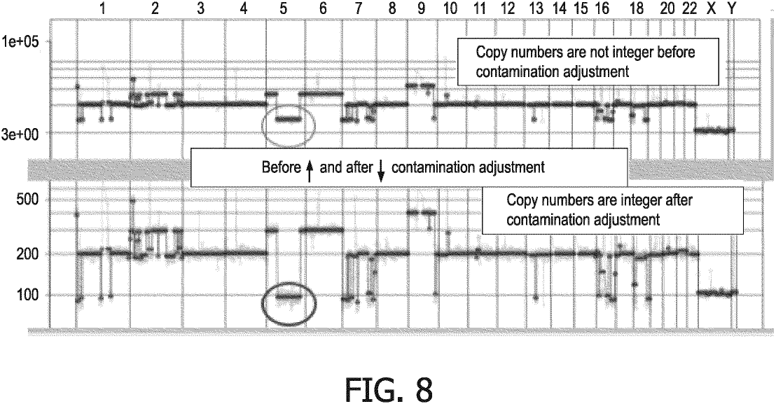

Referring to FIG. 8 is a comparison of an unadjusted CNV profile (top panel) and a generated best fit CNV profile (bottom panel). In the unadjusted CNV profile, the copy numbers are not integers due to contamination. See, for example, the circled copy number in the top panel. In the generated best fit CNV profile, the copy numbers are integers due to the process described or otherwise envisioned herein. See, for example, the circled copy number for the same segment in the bottom panel.

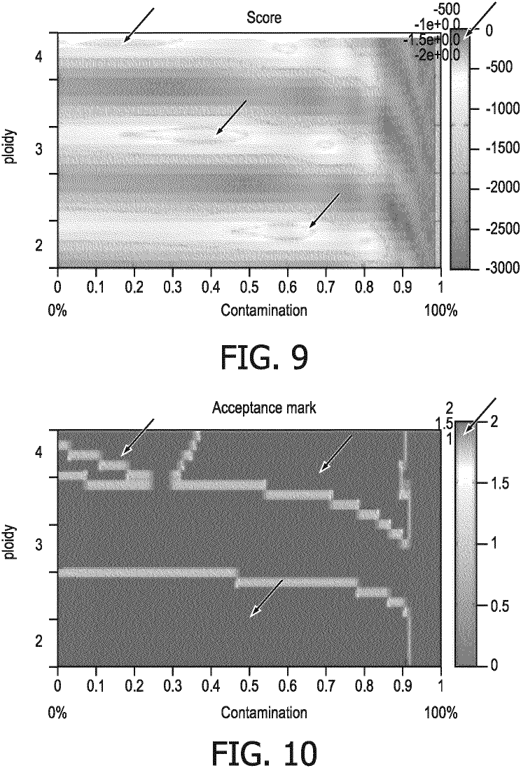

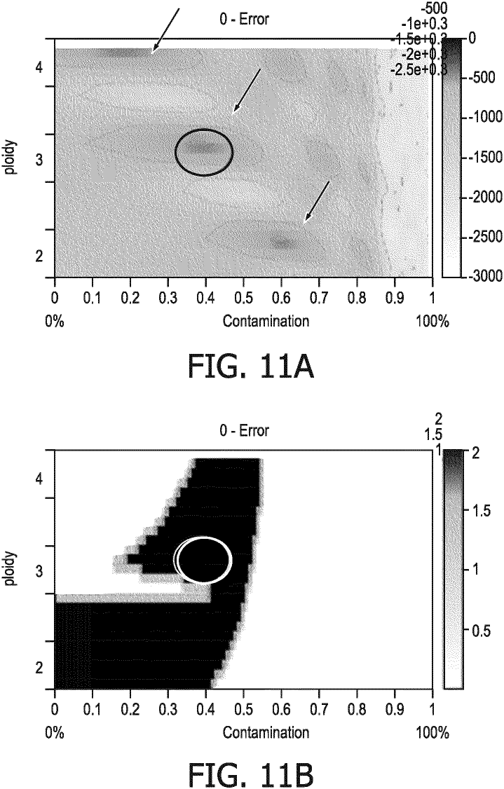

Referring to FIG. 9 is an example of a graph of adjustment score results for a given ploidy range (1.5 to 4) and a contamination range (0% to 100%), where the arrows show regions with favorable scores corresponding to the scale to the right of the graph. FIG. 10 is a graph of acceptable or preferred results for the given ploidy versus contamination ranges, where the arrows correspond to the more favorable results according to the scale to the right of the graph. The adjustment score shown by the arrow in the lower right side of the adjustment score graph in FIG. 9 corresponds to a preferred region in the acceptable or preferred results graph in FIG. 10, thus indicating the adjustment score as a possible best fit to generate a best fit CNV profile.

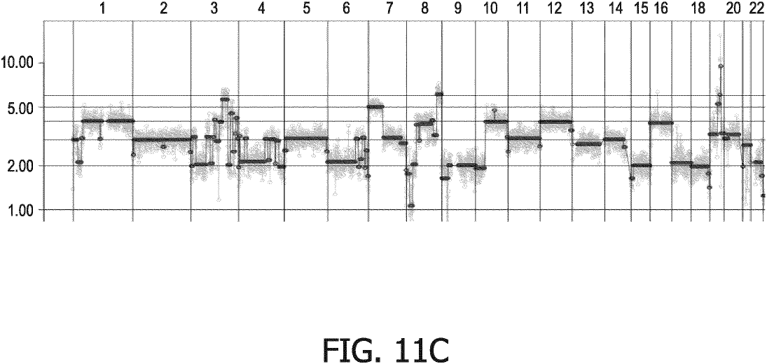

Similarly, referring to FIGS. 11A through 11C is an example of a best fit adjusted CNV profile selected using the methods and systems described or otherwise envisioned herein. FIG. 11A is a plot of adjustment scores for a ploidy range (1.5 to 4) and a contamination range (0% to 100%), where the arrows show regions with favorable scores, or in other words three potential solutions. FIG. 11B is a graph of acceptable or preferred results for ploidy versus contamination ranges. For example, the central region shown by the arrow corresponds to the more favorable result on the scale. The circled favorable score from FIG. 11A is selected as the best fit as it corresponds to a preferred result region in FIG. 11B, shown by the circled region in FIG. 11B. The other regions of favorable score from FIG. 11A, shown by the arrow in the upper left and the arrow in the lower right, do not correspond to preferred result regions in FIG. 11B. The selected best fit is then utilized to generate the best fit adjusted CNV profile in FIG. 11C.

At step 192 of the method, the system provides the generated adjusted CNV profile report. The report may comprise, for example, one or more of the original unadjusted CNV profile, the generated adjusted CNV profile, the received ploidy range (P) and interval, the received contamination rate (C) and interval, one or more calculated adjusted segmentation values, one or more calculated adjustment scores, a best fit adjustment score, information about the factor or factors that influenced selection of the best fit CNV profile, and/or other information. The report may be electronic or printed, and may be stored. For example, the report may comprise a text-based file or other format. The report may be sortable or otherwise configured for organization to allow easy analysis and extraction of information.

According to an embodiment, the CNV profiling system may visually display information about the generated adjusted CNV profile and/or any of the elements, scores, parameters, or factors described or otherwise envisioned herein. According to an embodiment, a clinician, researcher, or other user may only be interested in one piece of information such as the generated adjusted CNV profile, and thus the CNV profiling system may be instructed or otherwise designed or programmed to only display this information.

According to an embodiment, the report or information may be stored in temporary and/or long-term memory or other storage. Additionally and/or alternatively, the report or information may be communicated or otherwise transmitted to another system, recipient, process, device, and/or other local or remote location.

According to an embodiment, once the report or information is generated, it can be provided to a researcher, clinician, or other user to review and implement an action or response based on the provided information. For example, a researcher, clinician or other user may utilize the information to quantify clinically actionable CNVs based on the report as generated from sparse whole genome sequencing data. That this is generated from sparse whole genome sequencing data represents a novel and non-obvious improvement in the field, as prior studies teach away from this use either explicitly or by suggesting that sparse whole genome sequencing data is not data-rich or robust enough to provide the necessary amount of information.

Indeed, identifying causal CNVs can be an essential component of disease diagnosis and treatment. Clinically actionable CNVs present an important piece of information for disease, as well as a possible treatment point for disease. This is true not only in cancers but in many other disorders and phenotypes. For example, CNV evaluation can help improve diagnosis, monitoring, and treatment of neurological disorders. This may include scenarios where the neurological disorder is so rare that there is no diagnostic test in existence. In addition to neurological disorders, many other conditions may be diagnosed, monitored, and treated based on the identification of a causal CNV obtained by analysis of sparse whole genome sequencing.

Accordingly, at step 194 of the method, a user such as a healthcare professional or researcher receives a generated adjusted CNV profile report and identifies, based on the report, one or more causal CNVs for the phenotype. For example, the user may identify a causal CNV for a cancer or cancer phenotype, a neurological disorder, or any of a wide variety of other phenotypes. Also at step 194 of the method, the user identifies a treatment or other intervention for the individual based on the identified causal CNV, and applies that treatment to the individual. Notably, the identification of the CNV profile, the identification of an intervention, and the application of that intervention are based entirely on the ability of the CNV profiling system to generate an adjusted CNV profile using only the results of sparse whole genome sequencing. The use of sparse whole genome sequencing by the CNV profiling system has thereby significantly decreased cost, increased speed and efficiency of the CNV profiling system, and improved care of the individual.

According to another embodiment, a researcher, clinician or other user may utilize the information to quantify tumor purity, which may be a piece of information provided in the report or otherwise provided by the system. By determining a best fit for the CNV profile, the system is also thereby determining a purity, or rather the contamination, of the sample as measured by the initial unadjusted CNV profile. Many other downstream uses are possible.

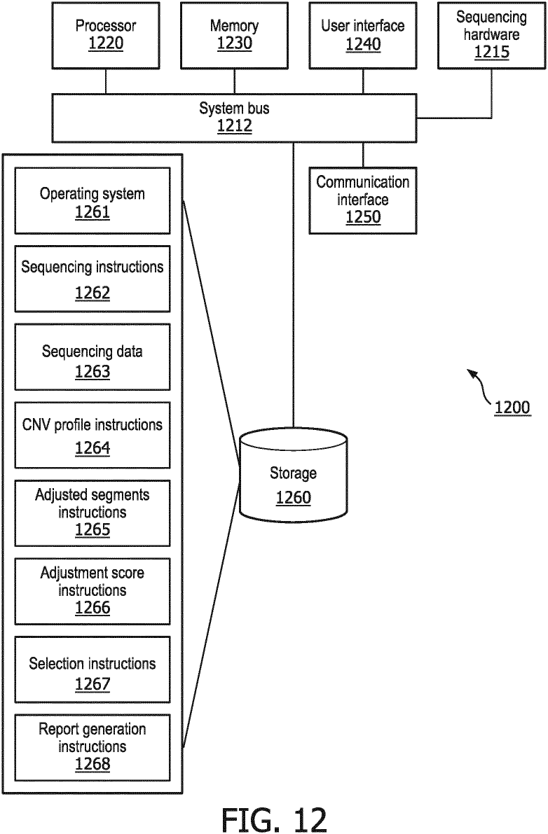

Referring to FIG. 12, in one embodiment, is a schematic representation of a CNV profiling system 1200 configured to determine copy number variation of tumor cells using sparse genome data and a CNV profiling system. System 1200 may be any of the systems described or otherwise envisioned herein, and may comprise any of the components described or otherwise envisioned herein.

According to an embodiment, system 1200 comprises one or more of a processor 1220, memory 1230, user interface 1240, communications interface 1250, and storage 1260, interconnected via one or more system buses 1212. It will be understood that FIG. 12 constitutes, in some respects, an abstraction and that the actual organization of the components of the system 1200 may be different and more complex than illustrated.

In some embodiments, such as those where the system comprises or directly implements a DNA sequencer or sequencing platform, the hardware may include additional sequencing hardware 1215. The sequencing platform is configured to generate sparse whole genome sequencing data from a sample. According to an embodiment, sparse whole genome sequencing data comprises much less information than high-depth next-generation whole genome sequencing data. For example, the sparse whole genome sequencing data may comprise fewer than 10 million reads for a human genome, comprising approximately 0.1x coverage of that genome.

According to an embodiment, system 1200 comprises a processor 1220 capable of executing instructions stored in memory 1230 or storage 1260 or otherwise processing data to, for example, perform one or more steps of the method. Processor 1220 may be formed of one or multiple modules. Processor 1220 may take any suitable form, including but not limited to a microprocessor, microcontroller, multiple microcontrollers, circuitry, field programmable gate array (FPGA), application-specific integrated circuit (ASIC), a single processor, or plural processors.

Memory 1230 can take any suitable form, including a non-volatile memory and/or RAM. The memory 1230 may include various memories such as, for example L1, L2, or L3 cache or system memory. As such, the memory 1230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 1200. It will be apparent that, in embodiments where the processor implements one or more of the functions described herein in hardware, the software described as corresponding to such functionality in other embodiments may be omitted.

User interface 1240 may include one or more devices for enabling communication with a user. The user interface can be any device or system that allows information to be conveyed and/or received, and may include a display, a mouse, and/or a keyboard for receiving user commands. In some embodiments, user interface 1240 may include a command line interface or graphical user interface that may be presented to a remote terminal via communication interface 1250. The user interface may be located with one or more other components of the system, or may located remote from the system and in communication via a wired and/or wireless communications network.

Communication interface 1250 may include one or more devices for enabling communication with other hardware devices. For example, communication interface 1250 may include a network interface card (NIC) configured to communicate according to the Ethernet protocol. Additionally, communication interface 1250 may implement a TCP/IP stack for communication according to the TCP/IP protocols. Various alternative or additional hardware or configurations for communication interface 1250 will be apparent.

Storage 1260 may include one or more machine-readable storage media such as read-only memory (ROM), random-access memory (RAM), magnetic disk storage media, optical storage media, flash-memory devices, or similar storage media. In various embodiments, storage 1260 may store instructions for execution by processor 1220 or data upon which processor 1220 may operate. For example, storage 1260 may store an operating system 1261 for controlling various operations of system 1200. Where system 1200 implements a sequencer and includes sequencing hardware 1215, storage 1260 may include sequencing instructions 1262 for operating the sequencing hardware 1215, and sparse whole genome sequencing data 1263 obtained by the sequencing hardware 1215, although sparse whole genome sequencing data 1263 may be obtained from a source other than an associated sequencing platform.

It will be apparent that various information described as stored in storage 1260 may be additionally or alternatively stored in memory 1230. In this respect, memory 1230 may also be considered to constitute a storage device and storage 1260 may be considered a memory. Various other arrangements will be apparent. Further, memory 1230 and storage 1260 may both be considered to be non-transitory machine-readable media. As used herein, the term non-transitory will be understood to exclude transitory signals but to include all forms of storage, including both volatile and non-volatile memories.

While CNV profiling system 1200 is shown as including one of each described component, the various components may be duplicated in various embodiments. For example, processor 1220 may include multiple microprocessors that are configured to independently execute the methods described herein or are configured to perform steps or subroutines of the methods described herein such that the multiple processors cooperate to achieve the functionality described herein. Further, where one or more components of system 1200 is implemented in a cloud computing system, the various hardware components may belong to separate physical systems. For example, processor 1220 may include a first processor in a first server and a second processor in a second server. Many other variations and configurations are possible.

According to an embodiment, storage 1260 of CNV profiling system 1200 may store one or more algorithms and/or instructions to carry out one or more functions or steps of the methods described or otherwise envisioned herein. For example, processor 1220 may comprise unadjusted CNV profile instructions 1264, adjusted segmentation values instructions 1265, adjustment score instructions 1266, selection instructions 1267, and reporting instructions 1268, among many other algorithms and/or instructions to carry out one or more functions or steps of the methods described or otherwise envisioned herein.

According to an embodiment, unadjusted CNV profile instructions or software 1264 direct the system to generate or determine an initial unadjusted CNV profile from the sparse genome data received or generated by the system, comprising a plurality of CNV calls for a plurality of chromosomes or other genomic regions or breakdowns. The initial unadjusted CNV profile determination may be performed using any of a wide variety of different CNV analysis platforms or methods.

According to an embodiment, the unadjusted CNV profile instructions or software may direct the system to further process the initial unadjusted CNV profile. For example, the instructions or software or other instructions or software may direct the system to normalize the unadjusted CNV profile in any of a wide variety of ways and methods, including existing normalization methods. According to an embodiment, the system may be configured to normalize the unadjusted CNV profile to a mean value of one.

According to an embodiment, adjusted segmentation values instructions or software 1265 direct the system to determine adjusted segmentation values for the plurality of CNV calls. The adjusted segmentation values may be determined in a variety of different ways and methods. According to an embodiment, the adjusted segmentation values instructions or software receive one or more input parameters for analysis. As examples, input can include a range for ploidy for the genome data, and/or receives a range for a contamination rate for the genome data. These ranges can be predetermined, including being pre-programmed or otherwise received by or provided to the system. According to an embodiment, a user such as a researcher, clinician, technician, or other user can provide the ranges as a setting, selection, or other information via a user interface or any other communication method. Thus, adjusted segmentation values may be determined using the unadjusted segmentation value for a CNV segment, a ploidy value based on input, and/or a contamination rate value based on input.

According to an embodiment, adjustment score instructions or software 1266 direct the system to determine a plurality of adjustment scores using adjusted segmentation values for the CNV segments. The adjustment scores may be determined in a variety of different ways and methods. According to one embodiment, an adjustment score measures, and may allow for the minimization of, the difference between an adjusted segmentation value and a whole integer closest to the value. For example, according to an embodiment the system may be designed such that CNV segments are likely to be clonal, meaning they are likely to be an integer such as 1, 2, 3, etc. rather than a value such as 1.4, 2.6, 3.1, etc. This may represent an underlying assumption that CNV segments are likely to be an integer. According to an embodiment, an adjustment score may be determined for an entire CNV profile, or the adjustment score may be determined for a sub-set of the CNV profile. This may be selected or otherwise determined by a user, may be selected or determined by the system, and/or may be selected or otherwise determined by other input or selection mechanism.

According to an embodiment, selection instructions or software 1267 direct the system to identify a best fit adjusted CNV profile. According to an embodiment, the combination of contamination rate (C) and ploidy estimate (P) that best minimizes error in the unadjusted CNV profile, and thus generates the most likely adjusted CNV profile, is selected as the best solution. According to an embodiment, if the tumor is a single copy number clone, the segments will fall very close to integer values when the contamination rate and tumor ploidy values are correct. Thus, the combination of contamination rate and tumor ploidy values that generate an adjustment score and adjusted CNV profile with the highest likelihood of accuracy is selected.

According to an embodiment, identifying a best fit adjusted CNV profile comprises comparison of the results of the adjustment score analysis to one or more factors to facilitate selection of a best fit CNV profile. This may result in one or more parameters or factors that may be used to select or influence selection of a best fit CNV profile, from among the profiles represented by the adjustment scores. The parameters or factors may include, for example, variables such as preferences such as likely CNV segment integers, among others. Other factors include ploidy distributions determined from previous data or analyses, and/or contamination distributions from previous data or analyses, including but not limited to analyses where one or more parameters of the sample or analysis were similar to the current sample or analysis. In other words, the system may utilize prior information to prioritize certain solutions. For example, the system may use the distribution of contamination rate or ploidy from similar samples obtained by other techniques.

According to an embodiment, selection instructions or software further direct the system to generate the best fit adjusted CNV profile using the selected adjustment. This can be performed by the system via a variety of methods and systems, to generate a final adjusted CNV profile that can be saved, reported, or otherwise stored or used by the CNV profiling system.

According to an embodiment, reporting instructions or software 1268 direct the system to generate a user report comprising information about the analysis performed by the system. For example, a report may comprise one or more of the original unadjusted CNV profile, the generated adjusted CNV profile, the received ploidy range (P) and interval, the received contamination rate (C) and interval, one or more calculated adjusted segmentation values, one or more calculated adjustment scores, a best fit adjustment score, information about the factor or factors that influenced selection of the best fit CNV profile, and/or other information.

The reporting instructions or software 1268 may direct the system to store the generated report or information in temporary and/or long-term memory or other storage. This may be local storage within system 1200 or associated with system 1200, or may be remote storage which received the report or information from or via system 1200. Additionally and/or alternatively, the report or information may be communicated or otherwise transmitted to another system, recipient, process, device, and/or other local or remote location.

The reporting instructions or software 1268 may direct the system to provide the generated report to a user or other system. For example, the CNV profiling system may visually display information about the best fit CNV profile and/or any other generated information on the user interface, which may be a screen or other display.

The CNV profiling system and approach described or otherwise envisioned herein enables a researcher, clinician, or other user to more accurately determine the CNV profile of the genetic sample, and thus to implement that information in research, diagnosis, treatment, and/or other decisions. This significantly improves the research, diagnosis, and/or treatment decisions of the researcher, clinician, or other user.

Notably, the methods and systems described herein comprise different limitations each comprising and analyzing millions of pieces of information. For example, sparse whole genome sequencing data comprises reads that number in the millions. Thus, analyzing the data to generate an initial CNV profile requires millions of points of information.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method determining a copy number variation (CNV) profile of target cells from a sample, using a CNV profiling system, comprising:
receiving sparse genome sequencing data comprising sequencing from both target and non-target cells from the sample;
determining, from the received sparse genome data, an unadjusted CNV profile comprising a plurality of CNV calls for a plurality of chromosomes;
normalizing the unadjusted CNV profile;
receiving a range for possible ploidy for the CNV profile, and/or receiving a range for a possible contamination rate for the CNV profile the contamination rate corresponding to contamination of the CNV profile by non-target cells;
determining, using the received ploidy range and/or received contamination rate range, adjusted segmentation values for the plurality of CNV calls;
determining a plurality of adjustment scores comprising a distance between adjusted segmentation values and closest whole integers for a CNV call;
comparing the determined plurality of adjustment scores to one or more predetermined factors for selecting a CNV profile best fit;
selecting, based at least in part on the comparison, one of the plurality of adjustment scores as a best fit for the copy number variation profile of the tumor cells of the tumor;
generating, using the selected a best fit adjustment score, an adjusted CNV profile report; and
reporting the generated adjusted CNV profile report,
wherein determining adjusted segmentation values for the plurality of CNV calls comprises determining an adjusted segmentation value for each CNV segment using a sampling rate for the ploidy range and/or for the contamination rate; and wherein the adjusted segmentation values are calculated using the equation:
S_{adj} = P(S - C)/(1 - C)
where S_{adj} is an adjusted segmentation value for a CNV segment, P is a ploidy value from the range for possible ploidy, C is a contamination rate value from the range for possible contamination rate, and S is a segmentation value before adjustment.

2. The method of claim 1, further comprising the step of identifying, using the CNV profile report, one or more causal CNVs and providing an intervention based on the identified one or more causal CNVs.

3. The method of claim 1, wherein the unadjusted CNV profile is normalized to a mean value of one.

4. The method of claim 1, wherein the range for possible ploidy for the CNV profile and the range for a possible contamination rate for the CNV profile is received from a user of the CNV profiling system.

5. (canceled)

6. The method of claim 1, wherein determining a plurality of adjustment scores comprises the equation
$$D = \sum {}_{i = 1}^{n} {( {S}_{a d j}^{i} - r o u n d \left({S}_{a d j}^{i}\right) )}^{2}$$
where D is a calculated distance between an adjusted segmentation value (S_{adj}) and a closest whole integer,
$${S}_{a d j}^{i}$$
is an adjusted segmentation value of an ith segment, and n is a number of autosome segments.

7. The method of claim 1, wherein one of the one or more predetermined factors for selecting a CNV profile best fit is a CNV profile, a ploidy value or range, and/or a contamination value or range previously observed and determined to be meaningful.

8. The method of claim 1, wherein the target cells are tumor cells.

9. A system for determining a copy number variation (CNV) profile of target cells from a sample, comprising:
sparse genome sequencing data comprising sequencing from both target and non-target cells from the sample;
a processor configured to: (i) determine, from the received sparse genome data, an unadjusted CNV profile comprising a plurality of CNV calls for a plurality of chromosomes; (ii) determine, using a received ploidy range and/or received contamination rate range, adjusted segmentation values for the plurality of CNV calls; where the contamination rate corresponds to contamination of the CNV profile by non-target cells (iii) determine a plurality of adjustment scores comprising a distance between adjusted segmentation values and closest whole integers for a CNV call; (iv) compare the determined plurality of adjustment scores to one or more predetermined factors for selecting a CNV profile best fit; (v) select, based at least in part on the comparison, one of the plurality of adjustment scores as a best fit for the copy number variation profile of the tumor cells of the tumor; and (vi) generate, using the selected a best fit adjustment score, an adjusted CNV profile report; and
a user interface configured to provide the generated report,
wherein (ii) determining adjusted segmentation values for the plurality of CNV calls comprises determining an adjusted segmentation value for each CNV segment using a sampling rate for the ploidy range and/or for the contamination rate; and wherein the adjusted segmentation values are calculated using the equation:
S_{adj} = P(S - C)/(1 - C) where S_{adj} is an adjusted segmentation value for a CNV segment, P is a ploidy value from the range for possible ploidy, C is a contamination rate value from the range for possible contamination rate, and S is a segmentation value before adjustment.

10. The system of claim 9, wherein the user interface is further configured to receive a range for possible ploidy for the CNV profile, and/or receive a range for a possible contamination rate for the CNV profile.

11. The system of claim 9, wherein the unadjusted CNV profile is normalized to a mean value of one.

12. (canceled)

13. The system of claim 9, wherein determining a plurality of adjustment scores comprises the equation
$$D = \sum {}_{i = 1}^{n} {( {S}_{a d j}^{i} - r o u n d \left({S}_{a d j}^{i}\right) )}^{2}$$
where D is a calculated distance between an adjusted segmentation value (S_{adj}) and a closest whole integer,
$${S}_{a d j}^{i}$$
is an adjusted segmentation value of an ith segment, and n is a number of autosome segments.

14. The system of claim 9, wherein one of the one or more predetermined factors for selecting a CNV profile best fit is a CNV profile, a ploidy value or range, and/or a contamination value or range previously observed and determined to be meaningful.

15. The system of claim 9, wherein the target cells are tumor cells.
